(19) United States
(12) Patent Application Publication
(10) Pub. No.: US 2024/0242844 A1
(43) Pub. Date: Jul. 18, 2024

(54) METHOD FOR MONITORING A PATIENT HAVING A NEUROLOGICAL AND/OR PROGRESSIVE DISEASE, SUCH AS MULTIPLE SCLEROSIS, PARKINSON'S DISEASE OR ALZHEIMER'S DISEASE

(71) Applicant: ORIKAMI GROUP B.V., Nijmegen (NL)

(72) Inventors: Johannes WEDA, Nijmegen (NL); Frank DE GRAAF, Nijmegen (NL); Pim Petrus Cornelius VAN OIRSCHOT, Nijmegen (NL)

(21) Appl. No.: 18/561,954

(22) PCT Filed: May 18, 2022
(86) PCT No.: PCT/NL2022/050267

§ 371 Date: Nov. 17, 2023

(30) Foreign Application Priority Data
May 19, 2021 (NL) 2028255

(51) Int. Cl. G16H 50/30; A61B 5/00

(52) U.S. Cl. CPC G16H 50/30; A61B 5/4076; A61B 5/7275; A61B 5/742

(57) ABSTRACT

The invention relates to a method for monitoring a patient having a neurological and/or progressive disease, such as multiple sclerosis, Parkinson's disease or Alzheimer's disease, which method comprises the steps of: —obtaining digital biomarker data points, at a regular basis, for example daily, weekly or monthly, from the patient by having the patient perform at least one test; —collecting the digital biomarker data points into a data set during a time period; —defining a state space model; —fitting the state space model with the data set; —graphically presenting the output of the fitted state space model over at least part of the time period in which the digital biomarker data points were collected into the data set. Optionally, the method is followed by deciding to start, stop or modify a medical treatment.

### FIELD OF THE INVENTION

The invention relates to a method for monitoring a patient having a neurological and/or progressive disease, such as multiple sclerosis, Parkinson's disease or Alzheimer's disease.

### BACKGROUND

In many neurological and/or progressive diseases patients suffer from cognitive and/or functional decline. One of such diseases is Multiple Sclerosis (MS). Typically in these diseases clinical tests have been designed for measuring and monitoring the disease state and disease progression. In practice these tests are typically performed under the supervision of a professional in a clinical setting and applied only once or twice per year.

Since these measures are often inherently noisy, they are mostly only useful for conclusions over a group of patients, such as in cross sectional studies, but not very useful in detecting clinical relevant change in individuals and thus make clinical decisions on an individual patient difficult. Recent literature on cognitive tests and walking tests have shown that the level of clinical improvement, which is relevant according to the literature (e.g. 10% for the symbol digit modalities test (SDMT), a cognitive processing speed measure, and 20% for walking tests like the timed 25 feet walking test (T25FWT)) is lower than the minimal detectable change of these measures (typical >30% on both the SDMT and T25FWT) and are obscured by the measurement noise.

Furthermore, these traditional methods are often burdened by several other considerations. First, excellent interobserver agreement is achieved only with extensive fitting. Second, the tests are (sometimes) time consuming, requiring about 5 minutes or longer per test for an experienced analyst. Furthermore, the test frequency (only once or twice per year) severely limits timely adjustment of treatment and detection of sudden relapses.

To overcome these problems, home monitoring solutions and digital biomarkers have been designed in recent years. Digital biomarkers are defined as objective, quantifiable physiological and behavioral data that are collected and measured by means of digital devices such as portables, wearables, implantables, or digestibles. For example, an individual's gait may be measured while completing a prompted action, such as the T25FWT. Such measurements can conveniently be performed by means of the inertial sensors that are usually present in mobile phones and the like. Often, these digital biomarkers try to mimic clinical tests but are intended to be performed by the patients themselves in or around their home environment. Since digital biomarkers are used in the field, the measurement variability can increase compared to the standard clinical test. However, that can be compensated by the much higher frequency of testing. Additionally, the higher testing frequency potentially provides more timely information on disease progression and allows for timely detection of health status, disease activity or relapses.

Frequent measurements of digital biomarkers themselves come with another set of problems:
Frequent application of digital biomarkers leads to learning or fitting effects, especially in tests in the cognitive domain;
Digital biomarkers sometimes add additional measurement variability compared to their traditional measurements, which makes it more difficult to use them in individualized decision making, for example to timely detect sudden changes such as relapses or discern slow trends over time, despite the higher measurement frequency.

Currently, in digital biomarkers visualizations, the measurements are simply shown as scatter plot or as bar chart sometimes accompanied with a smoothing line based on, for example, an average calculated using a moving window. Averaging is a well-known technique for reducing noise in frequent measurements to obtain better estimates of the mean. However, averaging approaches still have the following short-comings:
Averaging data points provide averages of the measurements, not necessarily of the true state of the patient—which is of interest to the doctors to adjust treatment.
Clearly, any averaging approach obfuscates any sudden change, making it impossible to detect relapses.
While an averaging approach may seem to capture long term trends, it is not possible to distinguish the long term trend from learning or fitting effects on the state of the patient.

### SUMMARY OF THE INVENTION

It is therefore an object of the invention to reduce or even remove the abovementioned disadvantages. This object is achieved with the method according to the invention. Accordingly, the invention relates to a method for monitoring a patient having a neurological and/or progressive disease, such as multiple sclerosis, Parkinson's disease or Alzheimer's disease, which method comprises the steps of:
obtaining digital biomarker data points, at a regular basis, for example daily, weekly or monthly, from the patient by having the patient perform at least one test;
collecting the digital biomarker data points into a data set during a time period;
defining a state space model;
fitting the state space model with the data set;
graphically presenting the output of the trained state space model over at least part of the time period in which the digital biomarker data points were collected into the data set.

### BRIEF DESCRIPTION OF THE DRAWINGS

FIG. 1 displays a schematical embodiment of the method according to the invention.

FIG. 2 shows a schematical presentation of the broader concept wherein the method of the invention can advantageously be applied.

FIG. 3 shows a first graphical representation of the output of the fitted state space model, obtained with the two minute walking test.

FIG. 4 shows a second graphical representation of the output of the fitted state space model, obtained with the symbol digit modalities test.

### DETAILED DESCRIPTION OF THE INVENTION

Elements in the figure are illustrated for simplicity and clarity and have not necessarily been drawn to scale. Further, the terms “first”, “second”, and the like in the present description and claims, if any, are generally used for distinguishing between similar elements and not necessarily for describing a sequential or chronological order.

In the context of the invention, by the term ‘patient’ is meant a recipient of health care services that are performed by healthcare professionals (e.g. a practitioner, a neurologist). The patient is most often ill, suffering from e.g. a neurological and/or progressive disease. Throughout the text, references to the patient will be made by male words like ‘he’, ‘him’ or ‘his’. This is only for the purpose of clarity and conciseness, and it is understood that female words like ‘she’, and ‘her’ equally apply.

In the context of the present invention, by digital biomarker data points are meant objective, quantifiable physiological and behavioral data points that are collected and measured by means of digital devices such as portables, wearables, implantables, or digestibles. Digital biomarker data points are an outcome of tests performed by a patient, such as the two minute walking test, six minute walking test, timed 25 feet walking test, visual response test, timed-up-and-go-test, (delayed) spatial-recall test, California Verbal Learning Test, Conveyer Belt, Sunshine, Viewpoint, Papyrinth and symbol digit modalities test. These tests are typically the standard tests that are conventionally performed in a non-digital manner. Thus, the digital biomarkers in the context of the invention are then equivalent to the outcomes of the tests that are performed in a non-digital manner.

The digital biomarkers used in a method of the invention may represent relevant MS symptoms that are selected based on their relevance in MS and on their relation with MS disease activity. Preferably, these digital biomarkers are cognitive processing speed and walking function.

In a method of the invention, the digital biomarker data points are obtained at a regular basis. This typically means that digital biomarker data are obtained at regular intervals, such as a day, a week or a month. These digital biomarker data can be regarded as sequential data, wherein data points are ordered into sequences by time. Typically, at least three digital biomarker data points are obtained in a method of the invention. In order to increase the accuracy of the output of the method, however, it is preferred, that the number of data points is much larger, for example at least 10, at least 25, at least 50, at least 100, or at least 500. The number of data points may also be 1.000 or less, 500 or less, 250 or less, 100 or less or 50 or less.

The term ‘state-space model’ is well-known in the art. It refers to a class of probabilistic graphical model that describes the probabilistic dependence between the latent state variable and the observed measurement. A skilled person knows how define a state space model and implement it in a given system.

By defining a state space model in a method of the invention and fitting the state space model with the data set, noise is filtered out, while a more accurate state is obtained than with averaging the data points.

So, with the fitted state space model it is possible to predict intermediate digital data points, filter digital biomarker data points caused by a faulty test, find the state and detect whether there is a sudden change in expected data points, for example caused by a relapse.

The data set is typically setup as an array. When, for example, the regular basis is daily then the array is made up from the first measurement (day 0) up to the last measurement (day X) with all days in between. Days without measurements are treated as missing values, while days with multiple measurements are combined into a single value, for example by averaging the values of the specific day.

In a preferred embodiment of the method according to the invention the state space model is a local linear trend model.

The local linear trend model tries to find the slope and intercept that give the best average fit to all the past data. Although the local linear trend model tends to deviate the most from the data at the end of the time series, where typically state space models are used for forecasting trends, it does provide an excellent fit for the historic data, which is the data a practitioner (for example a neurologist) of the patient will look at, when evaluating the progress of a neurological and/or progressive disease.

The local linear trend model is defined by the equations:

$${y}_{t} = {μ}_{t} + {ϵ}_{t} \\ {μ}_{t + 1} = {μ}_{t} + {v}_{t} + {ξ}_{t} \\ {v}_{t + 1} = {v}_{t} + {ζ}_{t}$$

Where γₜ are the observations, μₜ is the baselevel, and νₜ is the slope (trend). The ϵₜ, ξₜ and ζₜ are independently normally distributed errors.

With this local linear trend model the noise in the obtained digital biomarker data points can be reduced up to at least 75%.

As further elucidated below, data collection via smartphone enables a high measurement frequency. This allows the estimation of a day-to-day variation in the measurements. The local linear trend model is employed to distinguish this variation from changes in the underlying concept of interest level. This level is approximated for an individual patient, based on all measurements available at that moment (the model needs at least three measurements). The approximated level can, potentially in combination with that of other concept of interest levels, support clinical evaluation.

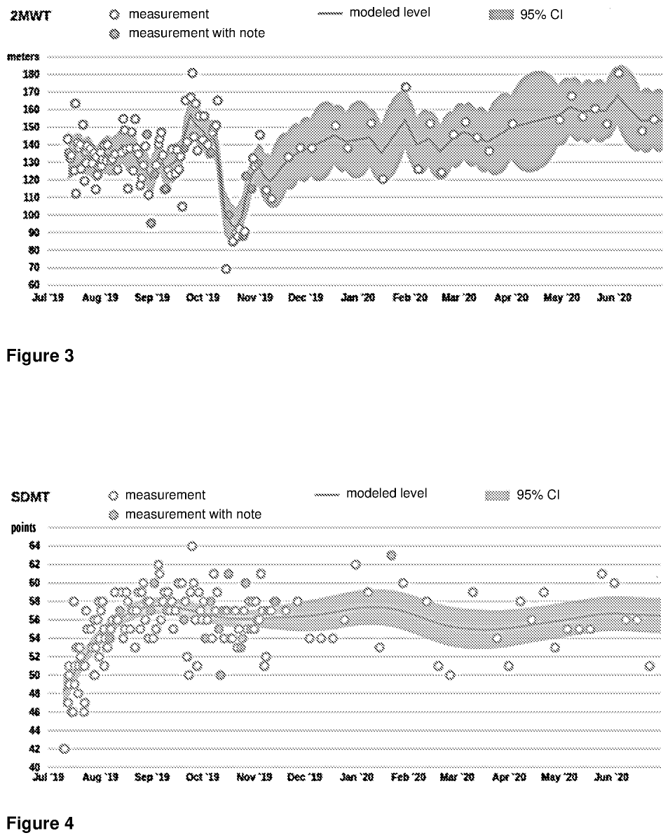

The level is typically shown as a line over time with a 95% confidence interval surrounding it (see e.g. FIG. 3 or 4). The model makes a few assumptions, such as a normal distributed day-to-day variation for a patient and that there is a certain relative effect in long-term changes (months or years) opposed to short-term changes (days or weeks). The confidence interval shows how accurate the model can estimate the level under these assumptions. It does not state what the probability distribution of the measurements is. Furthermore the correlation between the modelled level and the actual disease course can differ between patients.

A higher testing frequency leads to a more accurate output of the fitted state space model, and thus to a smaller 95% confidence interval surrounding it. This is also illustrated in FIGS. 3 and 4, where the testing frequency was decreased after October 2019—and as a result the 95% confidence interval widened thereafter.

It is for many patients however not desired to have a high testing frequency. Given their impaired health status, it is often too much for them to test daily, in particular to perform a daily walking test. Therefore, tests may be performed weekly rather than daily. Advantageously, the model can predict when a more accurate output is desired, requiring a temporary increase of testing frequency. Such prediction may be triggered when the model output identifies a particular medical condition, a physical change in the patient, a possible disease activity or a relapse.

Accordingly, a method of the invention may be followed by a step of changing a frequency of obtaining digital biomarker data points.

If desired a smoothing function, such as a spline, can be applied to the output of the trained state space model for graphically presenting said output. The smoothing function makes the curve calculated by the trained state space model more fluent, which helps in better finding trends in the obtained digital biomarker points and thus being able to better determine the progress of the neurological and/or progressive disease; and thus to be able to support conclusions on the health status of the patient.

In another embodiment of the method according to the invention a digital device such as a smartphone, portables, wearables, implantables, or digestibles, is used for obtaining digital biomarker data points from the patient and wherein the obtained biomarker data points are sent from the digital device to a server.

Such digital devices are for example provided with several sensors, such as touch sensors, heart beat sensors and accelerometers, which enables easy determination of the exercise a patient does during a test. Furthermore, the obtained data is easily sent to a server via the common internet connection present in such digital devices.

In a further embodiment of the method according to the invention the graphical representation of the output of the fitted state space model over at least part of the time period in which the digital biomarker data points is displayed on a second digital device, for example of the practitioner of the patient, connected to the server.

As the obtained digital biomarker data points are sent to a server, a second digital device can easily access such data and display the graphical representation to a practitioner. So, no direct or simultaneous contact needs to exist between patient and practitioner for monitoring a patient.

In yet a further embodiment of the method according to the invention the collected digital biomarker data points are included into the graphical presentation of the output of the fitted state space model. This allows for the practitioner to evaluate the actual data points or to evaluate the output of the state space model and to determine whether there are moments in time with unexpected values or relapses in the disease.

Thus, such graphical presentation provides immediate visual information representing the health status of the patient, from which valuable conclusions on the health status of the patient can be drawn. This enables the actual monitoring of the patient.

Accordingly, in an embodiment, the method of the invention is followed by monitoring the patient, which typically comprises giving the patient and/or his practitioner (eventually as a part of an entire professional healthcare team) personalized insight into the presence and progress of symptoms related to the patient's disease.

A graphical presentation as obtained in a method of the invention also enables personalized care and shared decision making (e.g. by patient and practitioner), for example decision making with respect to starting, stopping or modifying a medical treatment (such as applying a medication or performing another intervention). More generally, it enables workflow support to improve health outcomes for neurological diseases.

Accordingly, in an embodiment, the method of the invention is followed by making a decision on starting, terminating or modifying a medical treatment. This may concern a decision on how to proceed with an ongoing medical treatment of the patient, or on starting a new treatment of the patient.

In yet a further preferred embodiment of the method according to the invention the at least one test performed by the patient is selected from the group of: two minute walking test, six minute walking test, timed 25 feet walking test, visual response test, timed-up-and-go-test, (delayed) spatial-recall test, California Verbal Learning Test, Conveyer Belt, Sunshine, Viewpoint, Papyrinth and symbol digit modalities test.

These tests are known in the field and conventionally performed in the presence of the practitioner of the patient. However, with the method according to the invention, such presence is no longer necessary. This is advantageous since it allows that tests are performed at a higher frequency and in a trusted surrounding of the patient. Moreover, when a method of the invention is applied in e.g. the research, clinical practice or medical treatment of a neurological and/or progressive disease, this saves time of the patient as well as the practitioner. This for example concerns travel time, physical strains and/or emotional strains.

Another advantage of the method of the invention is that learning effects are compensated for in the output data, i.e. in the output of the fitted state space model. Especially in cognitive assessments, learning effects can become a significant factor in undermining the validity of the performed test.

These and other features of the invention will be elucidated in conjunction with the accompanying Figures.

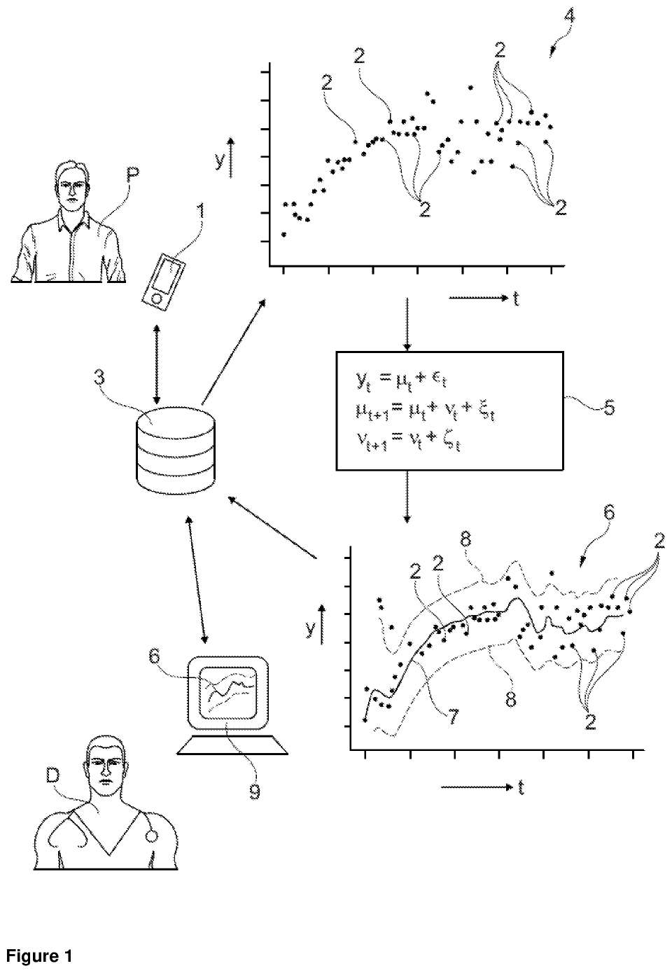

FIG. 1 shows a schematic embodiment of the method according to the invention. In the drawing a patient P is shown, which uses a smartphone (1) to obtain digital biomarker data points, for example by wearing the smartphone (1) during a test exercise or by doing a memory test on the smartphone (1). These digital biomarker data points (2) are stored in a database (3) as a data set (4), which is shown in the drawing as a scatter plot.

According to a method of the invention, the data set (4) is used to fit a local linear trend model (5). The output of this local linear trend model (5) is then graphically presented as a graph (6), which shows the modelled state (7) and a 80/90/95% confidence band of the state (8), as well as the digital biomarker data points (2).

The graphical presentation (6) can be stored on the server (3), such that at any desired moment a doctor (D) can look up the graph (6) on a computer (9) or any other suitable electronic device. The doctor D can quickly see from the graph (6) and the trend line (7) if the neurological and/or progressive disease of the patient P is progressing as expected or that any abnormalities occur.

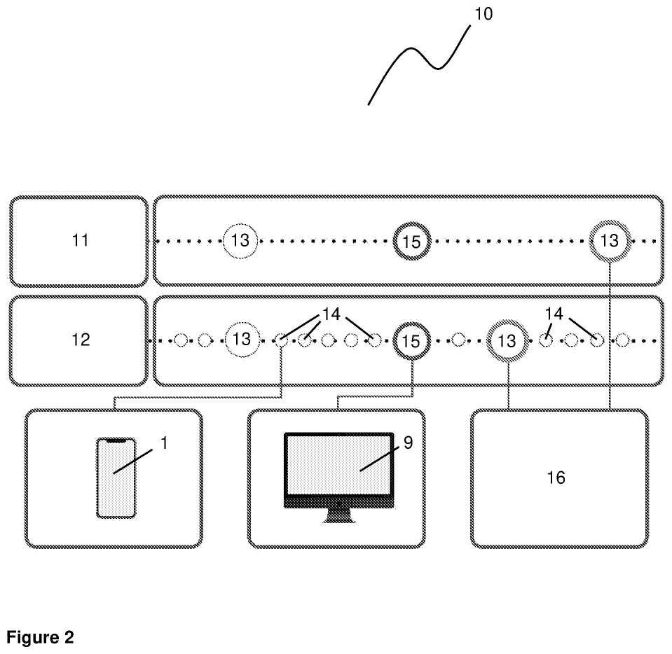

FIG. 2 shows a schematical presentation of the broader concept (10) wherein the method of the invention can advantageously be applied. Two timelines (11, 12) are displayed with events that occur during a period wherein the patient suffers from the disease, viz. a neurologist visit (13), instances wherein the patient takes tests at home (14), and a physical change in the patient (15), e.g. disease activity or relapse. The upper timeline (11) represents the course of events in conventional medical monitoring and treatment. The neurologist visits (13) are planned regularly, as there are no triggers to move up such visit (13). The lower timeline (12) represents the course of events in medical monitoring according to the invention. Associated with the lower timeline (12) are therefore a smartphone (1) to obtain digital biomarker data points; and a computer (9) to display the outcome of the model. It can be seen that the event of physical change in the patient (15) triggers a neurologist visit (13). This visit (13) falls earlier than the regular visit (13) contained in the upper timeline (11). This saves time (16), allowing earlier decision making which may lead to a better medical treatment for the patient.

FIGS. 3 and 4 each show a graphical representation of an output of a fitted local linear trend model, obtained with the two minute walking test (FIG. 3) and the symbol digit modalities test (FIG. 4). Each figure displays a graph, which shows the modelled level and a 95% confidence band thereof, as well as the individual measurements (i.e. the digital biomarker data points). The data were obtained during the monitoring of a particular MS patient (see also Examples).

### Examples

### 1. Conventional Clinical Practice

In the Netherlands, an MS patient is typically seen once or twice a year by a neurologist at the hospital or MS clinic, according to the current Dutch clinical practice. Typically, one or more of the following are performed: a neurological examination, an MRI scan, a (single) walking test and a cognitive assessment. The test scores are then administrated in the electronic patient file. FIG. 2 displays a timeline (11) of the events that occur in this conventional clinical practice.

The following is an (imaginary) case of an MS patient participating in such practice. During a visit to an MS clinic, he scores 140 meters on a two-minute-walking test (2MWT) and 54 points on a cognitive test (SDMT). During a second visit to the clinic approximately one year later, he expresses the feeling that his MS has gone worse, but this appears hard to quantify in the clinic. It is known that the smallest detectable changes of the clinical tests (˜35% of the measurement value) are larger than the clinically relevant changes, thus clinically relevant changes can only be detected if they are also larger than the smallest detectable change. Therefore, if the same tests were scheduled again, scores as small as 112 meter on the 2MWT or 49 points on the SDMT could still imply a stable situation. In addition, it is also the case that the patient has made a painful fall just after the first visit. Since this was not MS related, he does not mention this at the second visit. For the neurologist, it is then difficult to decide on the best treatment plan.

### 2. Clinical Practice According to the Invention

The method step of ‘obtaining digital biomarker data points’ is implemented on a smartphone that can be worn and operated by the MS patient, wherein the method step is performed in a mobile application that is installed on the smartphone (commonly known and hereinafter referred to as an ‘app’). This includes smartphone versions of the standard clinical tests (such as the 2MWT and the SDMT), so that these tests can easily and at a regular basis be performed in a domestic environment. Further, questions can be answered in the app, for example on the influence of MS on daily functioning or on the amount of pain. Personal notes can be made as well in the app. Patients and their caregivers jointly decide on the scheduling frequency of the tests, and this parameter can be set for each individual patient. Further, the smartphone comprises a dashboard designed to display (amongst others) the digital biomarker data and the output of the fitted state space model over a time period wherein the digital biomarker data points were obtained. The dashboard can also be displayed on a device used by the neurologist (for example a computer or a smartphone).

In ‘the cloud’, a local linear trend model is applied to the test data to estimate the daily level of the underlying state, and the trend with which this level is changing per day. If on a single day multiple tests are completed successfully and considered acceptable by the patient, the mean value of all measurements on that particular day is fed to the local linear trend model. The model can also estimate the daily level and trend if no measurements are performed that day, based on the test results of the past, in which more recently performed tests have a higher weighting than older test results. The confidence level with which this level can be estimated will be smaller when the assessment frequency is lower. However, with a weekly assessment frequency, changes as subtle as clinical relevant changes are can still be detected with statistical significance.

Thus, in the imaginary case described above in Example 1, if the MS patient would drop in score from 140 (range 130-150) meters to 112 (range 98-126) meters on the 2MWT, or from 54 (range 52-56) to 49 (range 47-51) points on the SDMT, this change can be detected with statistical significance when the app is used. Vice versa, if the estimated level is modelled not to have changed, a single outlier of 112 meters or 49 points would be visible in the dashboard, but outside of the estimated level with its 95% confidence interval (Cl) band.

The method of the invention is however also applied in a real case, wherein a real MS patient carries a smartphone on which the app is installed, and wherein the neurologist has access to the output of the fitted local linear trend model, which is displayed on his dashboard. The period wherein the MS patient has performed test spans July 2019-June 2020. FIGS. 3 and 4 represent a screenshot of the dashboard, which are graphical representations of the output of the fitted local linear trend model during this period, obtained with the SDMT and 2MWT, respectively. This patient (also) made a fall, which occurred in October 2019. This is visible as a significant drop in the distance walked during the 2MWT (while the output of the SDMT appears unaffected). After the fall, the estimated levels of the SDMT and 2MWT scores stay approximately the same in the period December 2019-June 2020, which aids the neurologist in the clinical decision making process, even though individual measurement values (without the application of the local linear trend model) are fluctuating and many individual data points of the SDMT curve fall outside the 95% Cl band around the estimated level in this example. FIGS. 3 and 4 further demonstrate that the testing frequency was substantially decreased after October 2019, leading to a wider 95% Cl band around the estimated level. FIG. 2 displays a timeline (12) of the events that occur in this clinical practice according to the invention.

1. Method for monitoring a patient having a neurological and/or progressive disease, such as multiple sclerosis, Parkinson's disease or Alzheimer's disease, which method comprises the steps of:
obtaining digital biomarker data points, at a regular basis, for example daily, weekly or monthly, from the patient by having the patient perform at least one test;
collecting the digital biomarker data points into a data set during a time period;
defining a state space model;
fitting the state space model with the data set;
graphically presenting the output of the fitted state space model over at least part of the time period in which the digital biomarker data points were collected into the data set.

2. Method according to claim 1, wherein the state space model is a local linear trend model.

3. Method according to claim 1, wherein a smoothing function, such as a spline, is applied to the output of the fitted state space model for graphically presenting said output.

4. Method according to claim 1, wherein a digital device such as portables, wearables, implantables, or digestibles, is used for obtaining digital biomarker data points from the patient and wherein the obtained biomarker data points are sent from the digital device to a server

5. Method according to claim 4, wherein the graphical representation of the output of the fitted state space model over at least part of the time period in which the digital biomarker data points is displayed on a second digital device, for example of the practitioner of the patient, connected to the server.

6. Method according to claim 1, wherein the collected digital biomarker data points are included into the graphical presentation of the output of the fitted state space model.

7. Method according to claim 1, wherein at least one test performed by the patient is selected from the group of: two minute walking test, six minute walking test, timed 25 feet walking test, visual response test, timed-up-and-go-test, (delayed) spatial-recall test, California Verbal Learning Test, Conveyer Belt, Sunshine, Viewpoint, Papyrinth, and symbol digit modalities test.

8. Method according to claim 1, wherein the graphically presented output of the state space model is the state and the 80/90/95% confidence interval of the state.

9. Method according to claim 1, wherein the method is followed by giving the patient and/or his practitioner personalized insight into the presence and/or progress of symptoms related to the patient's disease.

10. Method according to claim 1, wherein the method is followed by deciding to start, stop or modify a medical treatment.

11. Method according to claim 1, wherein the method is followed by changing a frequency of obtaining digital biomarker data points.
